(19) United States
(12) Patent Application Publication
(10) Pub. No.: US 2024/0173276 A1
(43) Pub. Date: May 30, 2024

(54) METHODS AND COMPOSITIONS FOR INHIBITING FORMATION OF THE HIV LATENT RESERVOIR

(71) Applicant: The University of North Carolina at Chapel Hill, Chapel Hill, NC (US)

(72) Inventors: David Margolis, Chapel Hill, NC (US); Edward Browne, Chapel Hill, NC (US)

(21) Appl. No.: 18/551,759

(22) PCT Filed: Mar. 22, 2022
(86) PCT No.: PCT/US2022/021259

§ 371 Date: Sep. 21, 2023

Related U.S. Application Data
(60) Provisional application No. 63/164,711, filed on Mar. 23, 2021.

(51) Int. Cl. A61K 31/167; A61K 45/06; A61P 31/18

(52) U.S. Cl. CPC A61K 31/167; A61K 45/06; A61P 31/18

(57) ABSTRACT

The present invention relates to methods for inhibiting human immunodeficiency virus (HIV) from entering latency in an infected subjected by co-administering an antiretroviral therapy regimen (ART) and a histone deacetylase (HDAC) inhibitor during a time window suitable for inhibiting entry into latency. The invention further relates to methods for treating HIV infections and compositions for carrying out the methods of the invention.

### STATEMENT OF PRIORITY

This application claims the benefit, under 35 U.S.C. § 119(e), of U.S. Provisional Application No. 63/164,711, filed on Mar. 23, 2021, the entire contents of which are incorporated by reference herein.

### STATEMENT OF GOVERNMENT SUPPORT

This invention was made with government support under AI126619 and AI143381 awarded by the National Institutes of Health. The government has certain rights in the invention.

### FIELD OF THE INVENTION

The present invention relates to methods for inhibiting human immunodeficiency virus (HIV) from entering latency in an infected subject by co-administering an antiretroviral therapy regimen (ART) and a histone deacetylase (HDAC) inhibitor during a time window suitable for inhibiting entry into latency. The invention further relates to methods for treating HIV infections and compositions for carrying out the methods of the invention.

### BACKGROUND OF THE INVENTION

Nearly 40 million people worldwide are infected with HIV, resulting in a million deaths each year. The development of antiretroviral therapy (ART) to treat HIV infection has been enormously successful at mitigating HIV disease and mortality, but interruption of therapy leads to rapid rebound of viremia due to a viral reservoir that persists during therapy. A key mechanism of HIV persistence is the ability of HIV to enter a state of virological latency in which viral gene expression is limited, allowing the provirus to remain invisible to the immune system. Latency within long-lived memory CD4 T cells permits extended persistence of the virus, and sporadic reactivation of these cells reignites viremia during treatment interruption. This reservoir is thus the main barrier to eradication of HIV in infected individuals.

Efforts to eliminate this reservoir have focused on the use of small molecules to reactivate viral gene expression (latency reversing agents, LRAs) thereby rendering infected cells vulnerable to clearance by the immune system. While this approach has achieved modest success at inducing viral gene expression in vitro and in vivo, this approach typically reactivates only a fraction of latent proviruses, and is insufficient to reduce the size of the reservoir. The limited efficacy of LRAs is likely due to the multiple layers of transcriptional and epigenetic repression that limit HIV proviral expression in infected resting CD4 T cells.

Given the difficulty in reducing the size of the persistent viral reservoir by reversing HIV latency and clearing infected cells in people on ART, the present invention addresses previous shortcomings in the art by providing new methods and compositions for preventing entry into latency during treatment of HIV infection.

### SUMMARY OF THE INVENTION

The present invention is based in part on the development of compositions and methods for inhibiting the entry of HIV into latency. Given the difficulty in eradicating HIV due to the latent reservoir of virus in infected subjects, preventing the formation of the majority of the latent reservoir would be an effective adjunct to current treatment.

In light of this, the inventors considered an alternative approach in which chemical agents (latency preventing agents, LPAs) are used to prevent actively infected CD4 cells from entering viral latency as they transition from an activated to a resting state. Such an approach has historically been considered clinically impractical due to observations suggesting that the reservoir is seeded very early during acute infection. However, it has recently been demonstrated by longitudinal analysis of HIV sequences during chronic infection that the majority (˜80%) of the reservoir is seeded at the time of ART initiation, suggesting that ART triggers changes to the viral or immune dynamics in infected individuals, leading to the formation or stabilization of a pool of latently infected cells. This discovery raises the possibility of a novel approach to targeting the reservoir, in which clinical dosing at the time of ART initiation with agents to prevent HIV from entering latency could be used to limit the size of the reservoir. Further, the inventors have determined that there is a small window of time during which administration of an LPA is effective. Administration outside of this window is minimally effective.

Accordingly, in one aspect, the invention relates to a method of inhibiting or preventing entry of human immunodeficiency virus (HIV) into latency, comprising administering to a cell comprising the HIV a fully suppressive antiretroviral therapy regimen (ART) and a histone deacetylase (HDAC) inhibitor.

Another aspect of the invention relates to a method of inhibiting or preventing entry of HIV into latency in a subject, comprising co-administering to the subject an ART and a HDAC inhibitor.

A further aspect of the invention relates to a method of treating HIV infection in a subject in need thereof, comprising co-administering to the subject an ART and a HDAC inhibitor, thereby inhibiting or preventing entry of HIV into latency and treating the subject.

An additional aspect of the invention relates to a composition comprising an ART and a HDAC inhibitor.

These and other aspects of the invention are set forth in more detail in the description of the invention below.

### BRIEF DESCRIPTION OF THE DRAWINGS

FIGS. 1A-1C show that vorinostat prevents HIV latency. A. Schematic showing primary CD4 T cell latency model design. B. Left panel shows emergence of a latent (GFP⁻) cell population from an initial GFP⁺ culture over time in the presence of a panel of small molecule inhibitors. Representative flow cytometry plots are shown in the right panel. Each datapoint represents the average of three replicates. C. HIV-eGFP infected cells were exposed to DMSO/vehicle control (D), continuous vorinostat (C), or pulsatile vorinostat—6 h per day (P). The percentage of GFP⁺ cells and the overall GFP intensity of the culture was measured over time. Each datapoint represents an average of three replicates.

FIGS. 2A-2B show transient vorinostat exposure leads to protracted viral gene expression. A. HIV-eGFP infected cells were exposed to different concentrations of vorinostat for 2 weeks, followed by two weeks in the absence of vorinostat. The percentage of latently infected cells (GFP⁻) at each time point was measured by flow cytometry. B. Vorinostat was either added at the beginning of a 3 week culture of HIV-GFP infected CD4 T cells then removed at times indicated (“wash out”, left panel) or added to the culture at various times post infection (“add in”, right panel). The overall percentage of latently infected cells (% GFP⁻) was measured at 3 wpi.

FIGS. 3A-3C show prolonged viral gene expression for a replication competent HIV strain by vorinostat. A. Schematic of experimental design. B. Flow cytometry of viral gene expression (HSA⁺) over time in the presence of vorinostat or DMSO. C. Representative flow cytometry from 6 dpi.

FIG. 4 shows vorinostat promotes an altered cellular phenotype. HIV-eGFP infected cells were cultured for 2 weeks in the presence of DMSO or vorinostat and analyzed by flow cytometry.

FIG. 5 shows dynamic expression of HDACs following CD4 T cell stimulation.

FIG. 6 shows HDAC3 is required for establishment of HIV latency in primary CD4 T cells. HIV-eGFP infected CD4 T cells were nucleofected with Cas9 complexed with sgRNAs targeting cellular class 1 HDACs or the viral Tat gene. The proportion of cells with active viral gene expression (GFP⁺) over time was monitored by flow cytometry (left panel). At 7 days post nucleofection, expression of HDAC1, HDAC2 and HDAC3 was examined by qPCR (right panel).

FIGS. 7A-7D show Class I HDAC selective inhibitors reveal druggable dependency on HDAC1 and 2 in latency initiation. A. Schematic overview of HDAC selective inhibitor experiments. HIV-GFP/Thy1.2 infected primary CD4 T cells were cultured in the presence of a dose curve of an inhibitor of HDAC1 and HDAC2 (HDAC1&2i), an inhibitor of HDAC3 (HDAC3i, Compound 38), vorinostat or control vehicle (DMSO 0.1%) for two weeks post infection and productive viral gene expression was examined by flow cytometry. B. Dose response curve showing the proportion of cells with productive viral gene expression after two weeks of treatment with the indicated inhibitors. Data shown are from one representative cell donor conducted in technical triplicate. C. Time course flow cytometry experiment in HIV-GFP/Thy1.2 infected primary CD4 T cells cultured with the indicated inhibitors. Vor: vorinostat 500 nM, HDAC1&2i 800 nM; HDAC3i: Compound 38, 3 μM. D. Mean fluorescent intensity (MFI) of GFP signal in Thy1.2+ cells in data from C. Data shown are representative of three independent experiments.

FIGS. 8A-8C show selective knockout of class I HDACs reveals genetic requirements for HDAC1 and HDAC3 in latency initiation. A. Schematic overview of HDAC CRISPR-experiment design. CD4 T cells were activated, infected with HIV-GFP/Thy1.2, then nucleofected with Cas9/sgRNA with either a non-targeting sgRNA or sgRNA directed against HIV-1 Tat, HDAC1, HDAC2, HDAC3, HDAC8, or both HDAC1 and HDAC2. B. Immunoblot of whole cell lysates from cells one week after sgRNA/Cas9 RNP nucleofection. C. Flow plot of productive viral gene expression measured in cells 21 days after sgRNA/Cas9 RNP nucleofection. Data shown are from three independent CD4 T cell donors infected in parallel, and is representative of three separate experiments.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, patent publications and other references cited herein are incorporated by reference in their entireties for the teachings relevant to the sentence and/or paragraph in which the reference is presented.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, 5%, ±1%, ±0.5%, or even ±0.1% of the specified amount.

The transitional phrase “consisting essentially of” means that the scope of a claim is to be interpreted to encompass the specified materials or steps recited in the claim, and those that do not materially affect the basic and novel characteristic(s) of the claimed invention.

The term “reactivate,” as used herein, refers to the activation of latent HIV proviruses present in resting CD4+ T cells to express viral genes, viral proteins, or viral particles.

The term “latent,” as used herein, refers to replication competent HIV proviruses present in resting CD4+ T cells that stably lack detectable expression of viral particles.

The term “reservoir,” as used herein, refers to the latent but replication competent HIV proviruses present in CD4⁺ T cells.

The term “antiretroviral therapy” or “ART” refers to a fully suppressive antiretroviral therapy regimen that comprises two or more antiretroviral drugs administered in one or more dosage forms, e.g., oral dosage forms and/or injectable dosage forms.

By the terms “treat,” “treating,” or “treatment,” it is intended that the severity of the subject's condition is reduced or at least partially improved or modified and that some alleviation, mitigation or decrease in at least one clinical symptom is achieved.

The terms “prevent,” “preventing,” and “prevention” refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective” amount as used herein is an amount that provides a desired effect.

A “treatment effective” or “therapeutically effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

One aspect of the invention relates to a method of inhibiting or preventing entry of human immunodeficiency virus (HIV) into latency, comprising administering to a cell comprising the HIV a fully suppressive antiretroviral therapy regimen (ART) and a histone deacetylase (HDAC) inhibitor.

Another aspect of the invention relates to a method of inhibiting or preventing entry of HIV into latency in a subject, comprising co-administering to the subject an ART and a HDAC inhibitor.

A further aspect of the invention relates to a method of treating HIV infection in a subject in need thereof, comprising co-administering to the subject an ART and a HDAC inhibitor, thereby inhibiting or preventing entry of HIV into latency and treating the subject.

Without being bound by theory, it is thought that the HDAC inhibitor blocks epigenetic changes that occur during the early stages of latency. The effect is to make infected cells at the time of ART more like cells prior to ART in which latency is less likely to occur. Further, the small window of time during which the HDAC inhibitor is effective in blocking entry into latency may relate to serial changes of cellular components which regulate the provirus, and serially silence proviral expression, making the proviral genome less responsive to the effects of HDAC inhibition. This may explain why HDAC inhibitors, which have been tested previously as LRAs after the initiation of ART, have not been found to be optimally effective, as the epigenetic changes associated with latency have occurred and the effect of local histone acetylation on proviral HDAC expression is marginal. While much attention has been paid to LRAs, latency prevention has not been much studied.

As used herein, co-administering refers to the administration of the ART and the HDAC inhibitor close enough together in time to elicit the benefits of the present invention, i.e., at about the same time. The initiation of administration of the ART and the HDAC inhibitor may be, for example, within 48 hours of each other, e.g., within 24, 18, 12, 10, 9, 8, 7, 6, 5, 4, 3, 2, or 1 hour of each other.

The period of co-administration of the ART and the HDAC inhibitor may be for any length of time suitable to inhibit HIV entry into latency. In some embodiments, the period of co-administration is about 2 weeks or less, e.g., about 1-2 weeks, e.g., about 1 week, e.g., 14, 13, 12, 11, 10, 9, 8, 7, 6, 5, 4, or 3 days or any range therein.

Generally, administration of the ART continues after administration of the HDAC inhibitor has ended, i.e., after the period of co-administration. Administration of the ART may continue for months or years as is known in the art.

The ART and the HDAC inhibitor may be administered in the same composition or in separate compositions. The ART and the HDAC inhibitor may be administered on the same schedule or different schedules. For example, the ART may be administered daily while the HDAC inhibitor is administered multiple times a day or the ART may be administered multiple times a day while the HDAC inhibitor is administered daily.

The methods of the invention may be effective to inhibit HIV entry into latency by at least 20%, e.g., at lest 30%, 40%, 50%, 60%, 70%, 80%, 90%, or 95% or more relative to an HIV-infected subject that has not undergone the methods of the invention. Measurement of the size of the latent reservoir may be carried out by methods well known in the art.

ART regimens that may be used in the methods of the invention are well known in the art. In some embodiments, the ART may be a combination anti-retroviral therapy (cART) or highly active antiretroviral therapy (HAART), e.g., at least two or three different agents from at least two different classes. Classes of agents that may be used in ART include, without limitation, reverse transcriptase inhibitors, protease inhibitors, viral integrase inhibitors, viral entry inhibitors, viral attachment inhibitors, viral maturation inhibitors, and any combination thereof. Individual agents that may be used in ART include, without limitation, saquinavir, atazanavir, darunavir, fosamprenavir, lopinavir, ritonavir, tipranavir, indinavir, ritonavir, nelfinavir, raltegravir, abacavir, bictegravir, tenofovir disoproxil fumarate, tenofovir alafenamide, elvitegravir, enfurvitide, emtricitabine, dolutegravir, fostemsavir, didanosine, stavudine, zidovudine, lamivudine, delavirdine, doravirine, rilpivirine, zalcitabine, nevirapine, efavirenz, etravirine, maraviroc, ibalizumab, cobicistat, and any combination thereof.

In one embodiment, the HDAC inhibitor is a class I HDAC inhibitor, e.g., a HDAC1, HDAC2, or HDAC3 inhibitor. In some embodiments, the inhibitor is specific for HDAC1, HDAC2, or HDAC3. In other embodiments, the inhibitor is effective against two or more HDACs, e.g., HDAC1 and HDAC2, e.g., a non-selective HDAC inhibitor. In some embodiments, the inhibitor inhibits the enzymatic activity of the HDAC. In some embodiments, the inhibitor targets the HDAC by means other than inhibition of enzymatic activity. For example, the HDAC, e.g., HDAC3, is targeted for degradation, e.g., using PROTAC technology or other degraders, or is structurally altered, e.g., by cleavage of the protein or binding by an agent that alters but does not cleave or degrade the HDAC. In some embodiments, the methods involve inhibition of the enzymatic activity of HDAC1 and/or HDAC2, optionally together with inhibition of HDAC3 by means other than inhibition of enzymatic activity. In some embodiments, more than one HDAC inhibitor is administered, e.g., 2, 3, 4, or 5 different HDAC inhibitors, e.g., each with different HDAC inhibitory activities. In some embodiments, the HDAC inhibitor is a small molecule, each having a molecular weight less than 1000 Da.

Examples of non-selective HDAC inhibitors or inhibitors of both HDAC1 and HDAC2 include, without limitation, vorinostat (SAHA), panobinostat (LBH589), trichostatin A (TSA), mocetinostat (MGCD0103), belinostat (PXD101), romidepsin (FK228, depsipeptide), givinostat (ITF2357), dacinostat (LAQ824), CUDC-101, quisinostat (JNJ-26481585) 2HCl, pracinostat (SB939), abexinostat (PCI-24781), AR-42, ricolinostat (ACY-1215), tacedinaline (CI994), fimepinostat (CUDC-907), M344, BRD3308, SR-4370, TC-H 106, NKL 22, tinostamustine (EDO-S101), UFO10, WT161, tucidinostat (chidamide), citarinostat (ACY-241), BG45, domatinostat (4SC-202), and HPOB.

Examples of selective HDAC1 inhibitors include, without limitation, entinostat (MS-275), MC1568, RG2833 (RGFP109), resminostat, suberohydroxamic acid, valproic acid (VPA), and splitomicin.

Examples of selective HDAC2 inhibitors include, without limitation, santacruzamate A (CAY10683).

Examples of HDAC3 inhibitors include, without limitation, droxinostat, RGFP966, isoguanosine, and tasquinimod.

Suitable subjects include mammals. The term “mammal” as used herein includes, but is not limited to, humans, non-human primates, bovines, ovines, caprines, equines, felines, canines, lagomorphs, etc. Human subjects include neonates, infants, juveniles and adults.

In some embodiments, the subject is a subject in need of treatment, e.g., a subject that has or is suspected of having an HIV infection or has been diagnosed with a disease or disorder associated with HIV infection, e.g., AIDS or AIDS-related complex. In some embodiments, the subject is an animal model of HIV infection, e.g., a rodent such as a mouse or a primate such as a monkey.

Another aspect of the invention relates to compositions useful for carrying out the methods of the invention. Some embodiments include a composition comprising an ART and a HDAC inhibitor that can be used during the co-administration part of the HIV treatment. In some embodiments, the composition is an oral dosage form (e.g., a tablet or capsule) or an injectable dosage form. In some embodiments, the composition is a unit dosage form.

The composition may be in a container in an amount sufficient for the co-administration period, e.g., a dosing period of about 2 weeks or less. One convenient container may be a blister pack containing sufficient unit dosage forms for the co-administration period. The blister pack may further comprise unit dosage forms for ART only to be used after the end of the co-administration period, suitably marked to be distinguishable from the compositions used during co-administration.

The compositions may comprise any of the ART agents or HDAC inhibitors described above.

As a further aspect, the invention provides pharmaceutical formulations and methods of administering the same to carry out the methods of the invention. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science and Practice of Pharmacy (21^{st} Ed. 2005). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo, comprising administering to a subject a pharmaceutical composition comprising a compound of the invention in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal, and inhalation administration, administration to the liver by intraportal delivery, as well as direct organ injection (e.g., into the liver, into the brain for delivery to the central nervous system, into the lymph nodes). In some embodiments, the formulation is delivered to the site of tissue damage (e.g., fibrosis) or inflammation. The most suitable route in any given case will depend on the nature and severity of the condition being treated and on the nature of the particular compound which is being used.

In certain embodiments, the inhibitor is administered via one or more of oral administration, injection, and a surgically implanted pump. In some embodiments, the administration is via intravenous injection, intraportal delivery, or direct liver injection.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein. The technology for forming liposomal suspensions is well known in the art. When the compound is in the form of an aqueous-soluble material, using conventional liposome technology, the same can be incorporated into lipid vesicles. In such an instance, due to the water solubility of the compound, the compound will be substantially entrained within the hydrophilic center or core of the liposomes. The lipid layer employed can be of any conventional composition and can either contain cholesterol or can be cholesterol-free. When the compound of interest is water-insoluble, again employing conventional liposome formation technology, the compound can be substantially entrained within the hydrophobic lipid bilayer which forms the structure of the liposome. In either instance, the liposomes which are produced can be reduced in size, as through the use of standard sonication and homogenization techniques. The liposomal formulations containing the compound disclosed herein, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In addition to compound, the pharmaceutical compositions can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions can contain microbial preservatives. Useful microbial preservatives include methylparaben, propylparaben, and benzyl alcohol. The microbial preservative is typically employed when the formulation is placed in a vial designed for multidose use. Other additives that are well known in the art include, e.g., detackifiers, anti-foaming agents, antioxidants (e.g., ascorbyl palmitate, butyl hydroxy anisole (BHA), butyl hydroxy toluene (BHT) and tocopherols, e.g., α-tocopherol (vitamin E)), preservatives, chelating agents (e.g., EDTA and/or EGTA), viscomodulators, tonicifiers (e.g., a sugar such as sucrose, lactose, and/or mannitol), flavorants, colorants, odorants, opacifiers, suspending agents, binders, fillers, plasticizers, lubricants, and mixtures thereof. The amounts of such additives can be readily determined by one skilled in the art, according to the particular properties desired.

The additive can also comprise a thickening agent. Suitable thickening agents can be those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products. Such thickening agents as described above can be included, e.g., to provide a sustained release effect. However, where oral administration is intended, the use of thickening agents as aforesaid will generally not be required and is generally less preferred. Use of thickening agents is, on the other hand, indicated, e.g., where topical application is foreseen.

In particular embodiments, the compound is administered to the subject in a therapeutically effective amount, as that term is defined above. Dosages of pharmaceutically active compounds can be determined by methods known in the art, see, e.g., Remington, The Science and Practice of Pharmacy (21^{st} Ed. 2005). The therapeutically effective dosage of any specific compound will vary somewhat from compound to compound, and patient to patient, and will depend upon the condition of the patient and the route of delivery. In one embodiment, the compound is administered at a dose of about 0.001 to about 10 mg/kg body weight, e.g., about 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9, or 10 mg/kg. In some instances, the dose can be even lower, e.g., as low as 0.0005 or 0.0001 mg/kg or lower. In some instances, the dose can be even higher, e.g., as high as 20, 50, 100, 500, or 1000 mg/kg or higher. The present invention encompasses every sub-range within the cited ranges and amounts.

The present invention is more particularly described in the following examples that are intended as illustrative only since numerous modifications and variations therein will be apparent to those skilled in the art.

### Example 1

### Experimental Procedures

Viruses and cell culture: Stocks of HIV-GFP were generated by co-transfection of 293T cells with the pNL4-3-Δ6-dreGFP plasmid (generous gift of Robert Siliciano) and the packaging plasmids PAX2-GagPol) and MD2-VSVG, using Mirus LT1 reagent. At 2 days post transfection virus was harvested from the supernatant and clarified by low speed centrifugation, followed by filtration through a 45 m filter.

Primary cell latency model: Primary CD4 T cells were isolated from fresh whole blood (purchased from Gulf Coast Regional Blood Center) by Ficoll isolation of peripheral blood mononuclear cells (PBMCs), then magnetic negative selection of CD4 T cells using a CD4 enrichment kit (Stem Cell). Total primary CD4 T cells were activated using anti-CD3/CD28 beads (Thermo Fisher) at a ratio of one bead per cell for 2 days, then infected with HIV-GFP viral supernatant by spinocculation for 2 hrs at 600 g with 4 g/mL polybrene. The infected cells were then resuspended in fresh RPMI with IL-2 (Peprotech) at 100 U/mL and incubated for two days before actively infected (GFP⁺) cells were sorted using a FACSAria flow sorter (Becton Dickson). The purified infected cells (GFP⁺) were then maintained at 1-2 million/mL with fresh media and IL-2 added every 2-3 days for 2 weeks. For latency reversing agent stimulation vorinostat was a generous gift from David Irlbeck (Viiv Healthcare), and was reconstituted in DMSO at 10 mM, before dilution into media to working concentrations of 125 nM-500 nM. Other small molecule inhibitors were purchased from commercial vendors.

Replication competent HIV infection: Stocks of replication competent HIV were prepared by transient transfection of 293T cells with a plasmid encoding a full length clone of NL4-3, with the Nef open reading frame replaced with the murine HSA (mCD24) gene. Cells infected with this virus are detectable by flow cytometry after staining with an anti-HSA-APC antibody. Primary CD4 T cells from healthy donors were activated with anti-CD3/CD28 beads for 3 days, then infected with viral supernatant and resuspended at 1 million cells per mL for 2 days, before flow cytometry to determine the level of infection. Anti-retroviral drugs (Raltegravir 1 μM and Abcavir 4 μM) were then added to prevent further viral spread, and the cells were cultured in the presence of control vehicle (DMSO 0.1%) or 500 nM vorinostat for 10 days. Vorinostat was then washed out of the culture and the cells culture for an additional 7 days. Samples were periodically removed for flow cytometry.

CRISPR/Cas9 nucleofection: CRISPR RNAs (crRNAs) targeting CTCF were pre-designed by IDT or designed with the BROAD institute GPP sgRNA designer. crRNAs and trans-activating RNAs (tracrRNA; IDT) were annealed in a thermocycler at a 1:1 ratio according to manufacturer's instructions and stored at −20° C. until use. Nucleofection experiments in primary CD4 T cells were performed six days post-infection using methods previously described for CRISPR nucleofection of either resting or activated human T cells. CRISPR-Cas9 ribonucleoprotein (RNP) complexes were generated by mixing crRNA:tracrRNA complexes with ALT-R S.p. Cas9 nuclease V2 at a 3:1 molar ratio for 10 minutes at room-temperature. Infected CD4 T cells were washed with PBS and 3 million cells per condition were resuspended in 20 μL buffer P2 (Lonza) with 4 μM IDT electroporation enhancer. CRISPR-Cas9 RNP nucleofection was performed with the EH100 nucleofection protocol on a 4D Nucleofector device (Lonza). Immediately after nucleofection cells were resuspended in fresh pre-warmed media containing 100 U/mL recombinant human IL-2.

Antibodies/Western blots: To extract protein, cells were washed in phosphate-buffered saline (PBS) then lysed in RIPA buffer (Sigma) supplemented with Complete protease inhibitors (Roche), before centrifugation to remove cell debris. Protein concentrations were then quantified using Bradford assay reagent (BioRad), and g of protein per sample was run on a 4-12% Tris Glycine polyacrylamide gel (Invitrogen). The gel was then transferred to a PVDF membrane. The membranes were then incubated in Tris-buffered saline with 0.1% Tween 20 (TBST) with a primary detection antibody for 2 hrs, before washing with TBST. Primary antibody staining was followed by incubation with an HRP-linked anti-rabbit antibody. Bands were then imaged by incubation of the membrane with ECL chemiluminescence reagents (ThermoFisher) and imaging on a ChemiDoc MP imager (BioRad).

Flow cytometry: For flow cytometry for murine Heat Shock Antigen (HSA), cells were washed in PBS before staining with anti-HSA-APC (Biolegend) at 1:200 dilution for 30 mins at 4° C., before washing in PBS and analysis using a Fortessa flow cytometer (Becton Dickson).

### Example 2

### Vorinostat Prevents HIV Latency

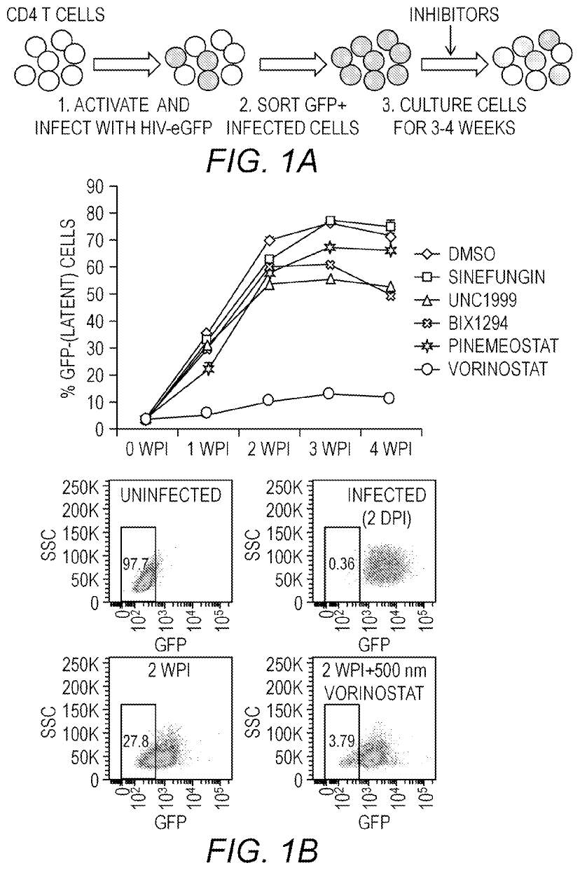

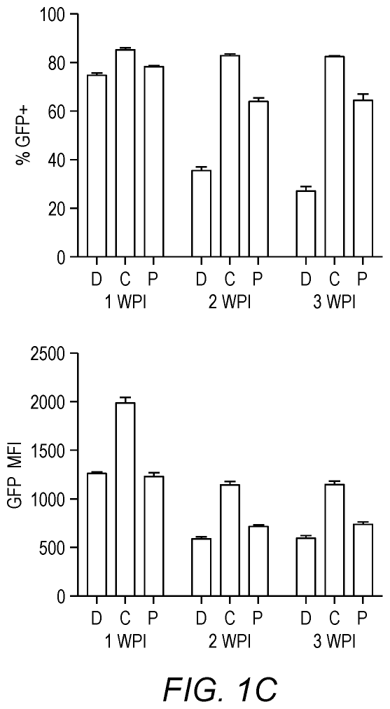

HIV latency is regulated by covalent modifications to provirus-associated histones that can promote or repress transcription. These modifications are mediated by a set of host cell enzyme complexes, including histone acetyl transferases (HATs), histone deacetylases (HDACs), histone methyltransferases (HMTs) and histone demethylases (HDMs). To investigate the role of these complexes in the establishment of HIV latency a primary CD4 T cell model of latency was examined. In this model, activated CD4 T cells are infected with a GFP expressing reporter strain of HIV. At 2 dpi, actively infected cells (GFP⁺) are sorted to obtain a pure actively infected population, then cultured for 3-4 weeks (FIG. 1A). During this period of culture, the infected cells progressively downregulate HIV gene expression, and a subset of cells become latently infected (GFP⁻). Importantly, these cells re-express HIV genes upon activation through their TCR, confirming that they are latently infected. To examine the role of specific histone modifying enzymes in this system, actively infected (GFP⁺) cells were isolated at 2 dpi and the cells cultured in the presence of a panel of inhibitors targeting chromatin modifying complexes. This panel included sinefungin (a pan-histone methyltransferase inhibitor), UNC1999 (an EZH2/H3K27 methyltransferase inhibitor), BIX1294 (a G9a/H3K9 methyltransferase inhibitor), pinometostat (a DOT1L/H3K79 methyltransferase inhibitor), and vorinostat (a histone deacetylase inhibitor). The infected cells were cultured in the presence of each inhibitor for up to 4 weeks. Each week post infection, the emergence of the latently infected (GFP⁻) population was measured by flow cytometry (FIG. 1B). As expected, vehicle (DMSO) treated cells exhibited progressive down-regulation of HIV gene expression during the period of culture, with up to 70% of cells becoming GFP⁻ by 3 weeks post infection (FIG. 1B). Notably, the inhibitors exhibited divergent effects on the formation of latently infected cells. Singefungin and pinometostat had no significant effect, while, at later timepoints (3-4 weeks post infection), UNC1999 and BIX1294 caused a modest reduction in the percentage of latently infected cells. By contrast, vorinostat potently inhibited the emergence of a latent (GFP⁻) population with a 7-fold reduction from 70% to 10% GFP In the presence of vorinostat, infected cells still exhibited an overall reduction of viral gene expression by 3 wpi compared to the starting population, indicating the presence of HDAC independent mechanisms of viral downregulation, but infected cells were prevented from becoming GFP⁻ and remained GFP^{lo} (FIG. 1B, right panel). These results identify vorinostat as a potent inhibitor of HIV latency establishment.

Previous clinical studies using vorinostat dosing have demonstrated rapid clearance, with concentrations peaking at 2-6 hours and declining 6-24 hours, raising the possibility that in vivo efficacy of vorinostat to prevent HIV latency could be limited by its pharmacokinetic properties. Thus, it was examined whether a daily transient pulsatile dose of vorinostat was also able to prevent HIV latency. Infected cells were exposed to either vehicle only (DMSO, 0.1%), continuous vorinostat (500 nM), or a daily 6 h pulse of vorinostat (500 nM) followed by an 18 h “wash-out” period. Infected cells in these three conditions were monitored for viral gene expression by flow cytometry for 3 weeks post infection (FIG. 1C). As previously observed, continuous vorinostat potently prevented the emergence of latently infected cells. For cells exposed to pulsatile vorinostat, it was observed that, although this dosing regimen was not as potent as continuous exposure to vorinostat, it was still able to significantly block the establishment of latency in a subset of cells.

### Example 3

### Transient HDACi Exposure Leads to Protracted Viral Gene Expression

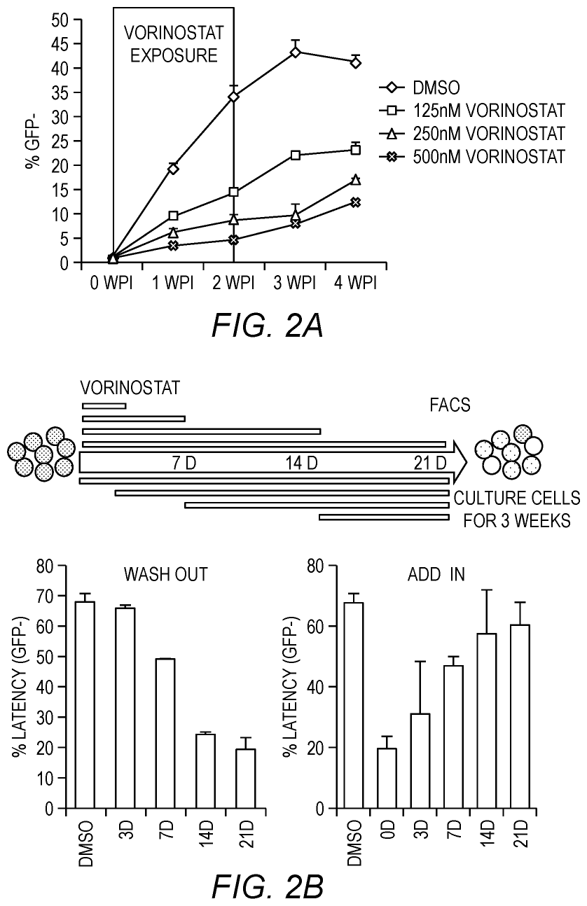

To further examine the impact of vorinostat on HIV gene expression after infection, activated HIV infected cells were exposed to a range of different vorinostat concentrations for two weeks, followed by an additional two week culture in the absence of vorinostat. As before, vorinostat potently blocked the establishment of latency. Furthermore, this response occurred in a clear dose-dependent fashion, with higher concentrations exhibiting more potent suppression of HIV latency (FIG. 2A). Remarkably, even after removal of vorinostat, the vorinostat-treated cells remained mostly GFP⁻ for the remaining 2 week period of culture. This finding suggests that a transient window of exposure of infected cells to vorinostat while these cells are transitioning from an activated state to a resting state can lead to extended viral gene expression post exposure. This phenomenon was further investigated by adding/removing vorinostat to/from the media at a range of timepoints post infection, in order to define the temporal window of susceptibility of HIV infected CD4 T cells to vorinostat (FIG. 2B). At three weeks post infection, the level of latently infected cells was measured by flow cytometry. The results indicated that vorinostat exhibited a significant impact on HIV latency if included the in the TC media between 0-14 days post infection. By contrast, addition or removal of vorinostat between 14 dpi and 21 dpi had little impact on the frequency of latently infected cells. These data suggest that HIV infected T cells exhibit a transient window of high susceptibility to vorinostat for 14 days after activation/infection followed by a relatively vorinostat resistant state after 14 days.

### Example 4

### Vorinostat Exposure Leads to Protracted HIV Gene Expression and Reduced Reservoir Seeding for a Replication Competent HIV Strain

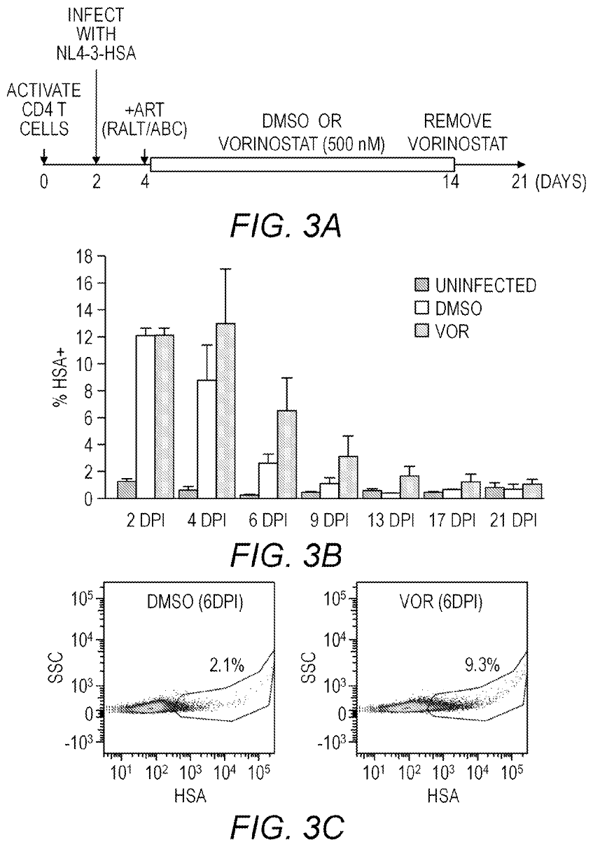

Since the primary cell model uses a defective reporter strain of HIV that lacks many accessory HIV genes, it was desirable to examine whether these observations were also applicable to a more complete replication competent strain of HIV. To address this question, activated CD4 T cells were infected with a modified NL4-3 strain of HIV with an HSA reporter cassette located within the Nef open reading frame. At 2 dpi anti-retroviral compounds (raltegravir and abacavir) were added to block further viral replication and the culture divided into parallel wells with either 500 nM vorinostat or with vehicle only (DMSO). Surface expression of HSA was monitored over time by flow cytometry. In this system, active viral gene expression is toxic to the infected cells, leading to a progressive decline in the percent HSA⁺ cells over time. Additionally, a subset of infected cells enters a latent state that can be revealed by subsequent reactivation. Consistent with the previous observations with the GFP-expressing reporter virus, vorinostat exposure resulted in significantly prolonged viral gene expression for at least 17 days after infection, suggesting that vorinostat may prevent or delay establishment of latency for intact full-length viral clones (FIGS. 3A-3C). This effect was most apparent at 4-6 dpi (2-4 days after beginning vorinostat dosing), but persisted after vorinostat was removed from the culture at 12 dpi. Interestingly, a small HSA⁺ population was still detectable for the vorinostat treated cells, even after vorinostat removal.

### Example 5

Vorinostat Exposure after CD4 T Cell Activation Promotes an Altered Cellular Phenotype

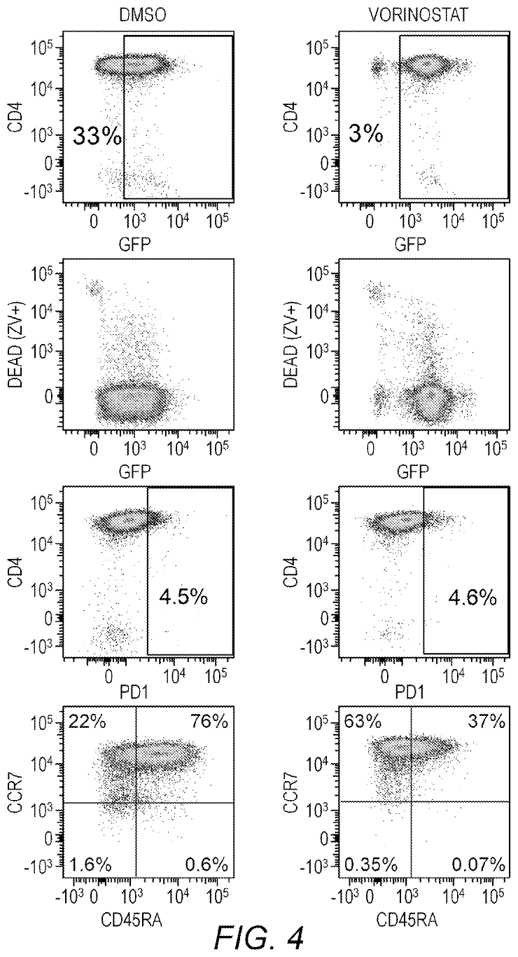

HIV gene expression is regulated by a set of host cell transcription factors that vary in activity as CD4 T cells adopt different activation states and sub-lineage identities post activation. It was hypothesized that vorinostat exposure post activation might promote altered expression of cellular activation markers or affect differentiation into specific CD4 T cell subsets. To investigate this possibility, flow cytometry was performed to examine the surface marker phenotype of HIV infected cells. Consistent with the hypothesis, it was observed that vorinostat significantly altered expression of some surface markers by 2-3 weeks post infection (FIG. 4). Specifically, an increased fraction of cells was observed that exhibited a central memory phenotype (CD45RA⁻ CCR7⁺) for cells that had been exposed to vorinostat post stimulation. Notably, vorinostat did not lead to increased expression of the CD4 T cell activation marker PD-1, indicating that vorinostat did not lead to general cellular activation.

### Example 6

### Dynamic HDAC Expression and Activity Following T Cell Activation

Vorinostat is a selective inhibitor for class I HDACs (HDAC1, HDAC2, HDAC3, and HDAC8). To further investigate the mechanisms of prolonged viral gene expression in the presence of vorinostat, the expression of class I HDACs was examined during T cell stimulation using anti-CD3/CD28 beads, and up to 3 weeks post stimulation, by western blot. Interestingly, these data showed clear dynamic regulation of HDAC expression during/post CD4 T cell stimulation. In particular, T cell activation caused a dramatic upregulation of HDAC2 expression, while HDAC1 and HDAC3 levels were largely unaffected (FIG. 5). Notably, after removal of the TCR-stimulating beads, all three HDACs exhibited a significant and progressive decline in expression as the cells returned to a resting state by 21 days post stimulation. These results indicate that recently activated CD4 T cells exhibit transient elevation of HDAC2 expression, followed by a gradual overall reduction in class 1 HDAC expression.

These dynamics of a transient increase in HDAC2 expression/activity followed by decreased HDAC levels/activity could potentially explain the high sensitivity of HIV gene expression to vorinostat soon after activation and the subsequent resistance of HIV in resting CD4 T cells to vorinostat. During a period of high HDAC activity and active removal of acetyl groups post stimulation, an HDAC inhibitor will likely have a more profound impact on the overall steady state levels of histone associated acetyl groups. By contrast, in resting post-activated cells, low HDAC expression and slow removal of acetyl groups will likely render the provirus relatively resistant to an HDAC inhibitor.

### Example 7

### HDAC3 is Required for HIV Latency Establishment

Due to the ability of an HDAC inhibitor, vorinostat, to prevent establishment of HIV latency, it was hypothesized that specific host cell targets of vorinostat are required to establish latency. Vorinostat most potently inhibits members of the class I HDAC subfamily (HDAC1, HDAC2, HDAC3, and HDAC8). To examine the role of individual HDACs in this model system, CRISPR/Cas9 mutagenesis of HDAC1, 2, and 3 was performed in HIV infected CD4 T cells. Primary CD4 T cells were first activated by TCR stimulation for 2 days, then infected with HIV-GFP. At 2 dpi, HIV infected CD4 T cells were then nucleofected with Cas9 protein/sgRNA complexes that targeted HDAC1, HDAC2, or HDAC3. A non-targeting sgRNA, and an sgRNA targeting the viral transcription factor Tat were used as controls. To confirm target specific knockout of individual HDACs, real-time PCR was performed for all three targets. As expected, each HDAC exhibited significantly reduced expression only in conditions in which that HDAC was targeted. Knockdown efficiencies ranging from 50% to 75%.

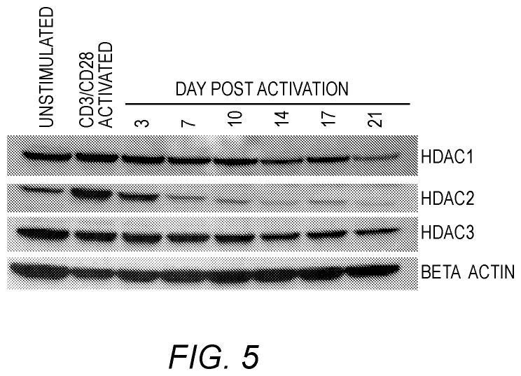

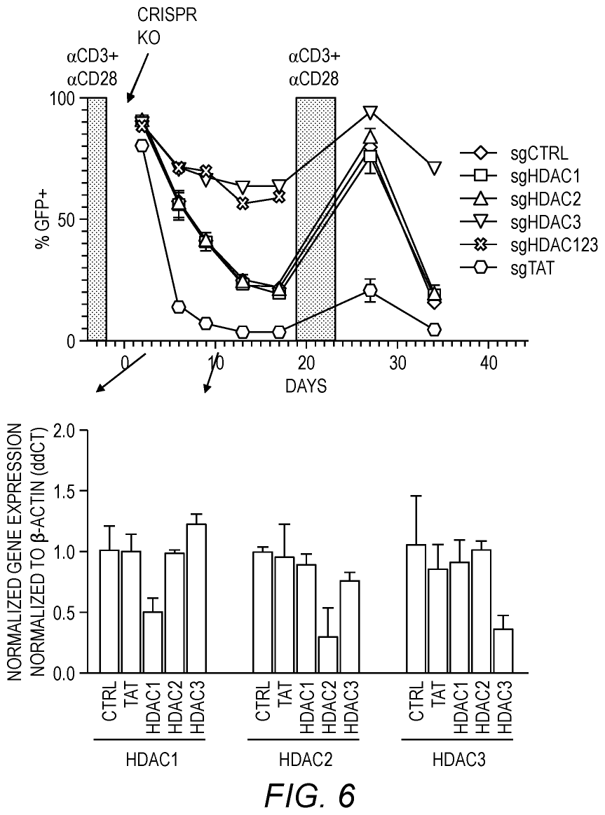

The impact of each sgRNA on the establishment of HIV latency was examined. The cell culture was examined by flow cytometry at 1, 2, and 3 weeks post infection, and the fraction of cells with active viral gene expression (GFP⁺) was measured. Notably, the Tat-specific sgRNA led to rapid loss of HIV gene expression in the culture, confirming that this experimental system was sensitive to perturbations in factors regulating HIV transcription (FIG. 6). Knockout of HDAC1 and HDAC2 had no detectible effect on HIV latency compared to a non-targeting sgRNA. Interestingly, sgRNAs targeting HDAC3 strongly inhibited the development of a latently infected (GFP⁻) population. HDAC3 depleted cells maintained ˜70% GFP⁺ cells at 18 days post targeting, while control cultures had ˜20% GFP⁺. To further examine this phenomenon, the cells were restimulated through their TCR at 19-22 dpi. For all the HDAC targeted cultures and control sgRNA culture, GFP was re-expressed in the majority of cells. Tat targeted cells, by contrast exhibited minimal viral response to TCR stimulation. After TCR-stimulating beads were removed, the HDAC1/2 targeted cells again rapidly reestablished latency, while HDAC3 targeted cells maintained a high % of GFP⁺ cells. These data indicate the HDAC3 plays a key role on the establishment of HIV latency.

### Example 8

### HDAC1 and HDAC3 Play Essential and Distinct Roles in HIV Silencing in CD4 T Cells

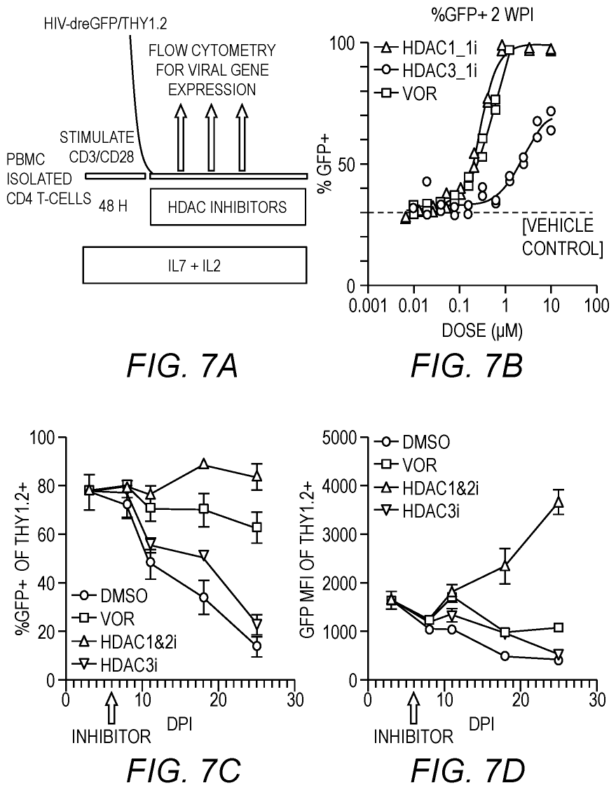

It was desirable to identify the cellular targets by which vorinostat prevents HIV silencing in CD4 T cells during effector to memory transition. Vorinostat inhibits Class I HDACs (HDAC1, HDAC2, HDAC3 and HDAC8). However, the contribution and roles of individual HDACs are largely unknown. Because of the high homology of HDAC1 and HDAC2 in the enzyme activation site, selective inhibitors for HDAC1 or HDAC2 have not been developed. Thus, the impact of novel small molecules that inhibit both HDAC1 and HDAC2 (HDAC1/2i) or HDAC3 (HDAC3i) on HIV silencing in CD4 T cells was examined. Activated CD4 T cells were infected with HIV-dreGFP/Thy1.2 and cultured for 14 d in the presence of each inhibitor, and viral gene expression was measured by flow cytometry (FIG. 7A). Importantly, dose-responsive latency prevention was observed with the HDAC1/HDAC2i with a similar potency to vorinostat, indicating a key role for HDAC1 or HDAC2 (FIG. 7B). By contrast, the HDAC3i (compound 38) prevented HIV silencing to a much lower extent (FIG. 7B), a result confirmed using a structurally distinct HDAC3i (RGFP966). Remarkably, for HDAC1/HDAC2i-treated cells, a progressive increase in viral gene expression intensity was also observed over time, significantly beyond that induced by vorinostat (FIG. 7D). Indeed, HDAC1/HDAC2i-treated cells had an approximately two-fold higher GFP intensity than cells immediately after activation and infection, the typical point of maximal GFP signal. This result suggests that specific targeting of HDAC1 and HDAC2 may be more effective than a broader block to class 1 HDACs with respect to preventing HIV silencing in CD4 T cells.

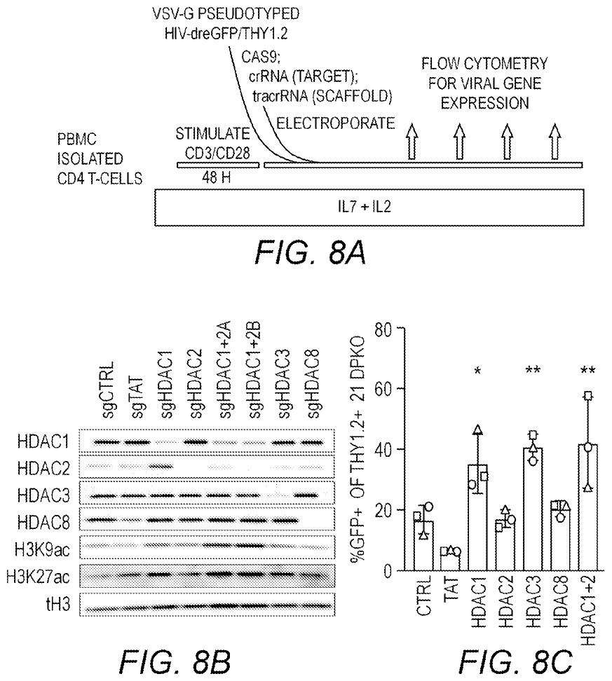

It was next sought to further validate the roles of individual HDACs in this system by using CRISPR/Cas9 targeting to knockout individual Class I HDACs in HIV-infected CD4 T cells (FIG. 8A). Conditions were optimized for CD4 T-cell nucleofection with sgRNA/Cas9 ribonucleoprotein complexes, enabling robust depletion of HDAC1, HDAC2, HDAC3, HDAC8, or of both HDAC1 and HDAC2 by 7 days after nucleofection, as measured by immunoblot (FIG. 8B). HDAC2 expression increased after HDAC1 knockout, likely reflecting a regulatory feedback loop (FIG. 8B). Interestingly, knockout of HDAC1 or HDAC3 significantly attenuated viral silencing, whereas targeting HDAC2 or HDAC8 had no impact (FIG. 8C). Therefore, both HDAC1 and HDAC2 were targeted with CRISPR/Cas9 in combination and individually. Significantly, a larger impact on proviral silencing was observed in the context of HDAC1/HDAC2 double knockout than for either individual knockout. Taken together, the inhibitor and genetic data suggest that HDAC1 and HDAC3 play essential but mechanistically distinct roles in latency initiation, and HDAC2 can partially compensate for HDAC1 loss because of overlapping functional roles. The redundancy of HDAC 1 and 2 in HIV LTR regulation has previously been described in other contexts. Given the clear impact of HDAC3 CRISPR/Cas9 knockout, the relatively modest effect of an HDAC3i was surprising. It is speculated that HDAC3 may either have an enzymatic role in latency establishment that is incompletely inhibited by chemical inhibition or that HDAC3 may play a non-enzymatic role. Notably, non-catalytic roles for HDAC have been previously reported. Overall, these data demonstrate that HDAC1 and HDAC2 together play an essential catalytic role in viral silencing for which these proteins are individually redundant, while HDAC3 plays an essential but potentially non-catalytic role. Thus, multiple HDACs contribute distinct activities to the process of HIV latency initiation.

The foregoing is illustrative of the present invention, and is not to be construed as limiting thereof. The invention is defined by the following claims, with equivalents of the claims to be included therein.

1. (canceled)

2. A method of inhibiting or preventing entry of human immunodeficiency virus (HIV) into latency in a subject, comprising co-administering to the subject an antiretroviral therapy regimen (ART) and a histone deacetylase (HDAC) inhibitor.

3. A method of treating HIV infection in a subject in need thereof, comprising co-administering to the subject an ART and a HDAC inhibitor, thereby inhibiting or preventing entry of HIV into latency and treating the subject.

4. The method of claim 3, wherein administration of the ART and the HDAC inhibitor starts at about the same time.

5. The method of claim 3, wherein the HDAC inhibitor is administered for a period of about 2 weeks or less.

6. The method of claim 3, wherein administration of the ART continues after administration of the HDAC inhibitor has ended.

7-8. (canceled)

9. The method of claim 3, wherein the HDAC inhibitor is a class I HDAC inhibitor.

10. The method of claim 3, wherein the HDAC inhibitor inhibits the enzymatic activity of HDAC1 and/or HDAC2.

11. The method of claim 3, wherein the HDAC inhibitor degrades or structurally alters HDAC3.

12. The method of claim 3, wherein the HDAC inhibitor is vorinostat.

13. The method of claim 3, comprising administering more than one HDAC inhibitor.

14. The method of claim 3, wherein the ART is selected from the group consisting of reverse transcriptase inhibitors, protease inhibitors, viral integrase inhibitors, viral entry inhibitors, viral attachment inhibitors, viral maturation inhibitors, and any combination thereof.

15. The method of claim 14, wherein the ART is selected from the group consisting of saquinavir, atazanavir, darunavir, fosamprenavir, lopinavir, ritonavir, tipranavir, indinavir, ritonavir, nelfinavir, raltegravir, abacavir, bictegravir, tenofovir disoproxil fumarate, tenofovir alafenamide, elvitegravir, enfurvitide, emtricitabine, dolutegravir, fostemsavir, didanosine, stavudine, zidovudine, lamivudine, delavirdine, doravirine, rilpivirine, zalcitabine, nevirapine, efavirenz, etravirine, maraviroc, ibalizumab, cobicistat, and any combination thereof.

16-18. (canceled)

19. A composition comprising an ART and a HDAC inhibitor.

20-21. (canceled)

22. The composition of claim 19, wherein the HDAC inhibitor is a class I HDAC inhibitor.

23. The composition of claim 19, wherein the HDAC inhibitor inhibits the enzymatic activity of HDAC1 and/or HDAC2.

24. The composition of claim 19, wherein the HDAC inhibitor degrades or structurally alters HDAC3.

25. The composition of claim 19, wherein the HDAC inhibitor is vorinostat.

26. The composition of claim 19, comprising two or more different HDAC inhibitors.

27. The composition of claim 19, wherein the ART is selected from the group consisting of reverse transcriptase inhibitors, protease inhibitors, viral integrase inhibitors, viral entry inhibitors, viral attachment inhibitors, viral maturation inhibitors, and any combination thereof.

28. The composition of claim 27, wherein the ART is selected from the group consisting of saquinavir, atazanavir, darunavir, fosamprenavir, lopinavir, ritonavir, tipranavir, indinavir, ritonavir, nelfinavir, raltegravir, abacavir, bictegravir, tenofovir disoproxil fumarate, tenofovir alafenamide, elvitegravir, enfurvitide, emtricitabine, dolutegravir, fostemsavir, didanosine, stavudine, zidovudine, lamivudine, delavirdine, doravirine, rilpivirine, zalcitabine, nevirapine, efavirenz, etravirine, maraviroc, ibalizumab, cobicistat, and any combination thereof.
